(19) United States
(12) Plant Patent Application Publication
(10) Pub. No.: US 2008/0320628 P1
(43) Pub. Date: Dec. 25, 2008

(54) Variety of Phormium plant named 'FIT01"

(76) Inventor: Pat FitzGerald, Co Kilkenny (IE)

Correspondence Address: FULWIDER PATTON LLP, 6060 CENTER DRIVE, 10TH FLOOR, LOS ANGELES, CA, 90045, US

(21) Appl. No.: 11/820,264

(22) Filed: Jun. 19, 2007

(51) Int. Cl. A01H 5/00

(52) U.S. Cl. PLT/263.1

(57) ABSTRACT

A new and distinct cultivar of Phormium cookianum plant named ‘FIT01’ characterized by shiny, deep blackish purple leaves, a taller and wider growth habit with wider leaves, and a robust appearance in adverse conditions.

### BACKGROUND OF THE NEW PLANT

The present invention relates to a new and distinct cultivar of Phormium plant, botanically known as Phormium cookianum, and further known by the varietal name ‘FIT01’. This new variety is also sold under the trade name Black Adder (Breeder's Ref. No. FIT01). The new variety was created through tissue culture mutation in Enniscorthy, Ireland, from the variety Phormium cookianum “Platts Black.” This variety was selected from a number of different mutations in a tissue laboratory in March 2003. Five different mutations were isolated for possible development, from which the present invention was chosen for further multiplication production. In January 2005, the cultures of Black Adder were further multiplied and the first crop began to develop. The variety was grown in both pots and one large original plant remains in situ outdoors. All plants were grown in Kilkenny, Ireland. For purposes of this application, the plants were evaluated outdoors and indoors in a plastic green house.

Following selection of the plantlet, the cultivar was propagated by tissue culture. Continued observation of future generations have confirmed that the distinguishing features of this new cultivar came true, remain stable and are retained through successive propagation.

The closest comparator to ‘FIT01’ known to applicant is Phormium cookianum Platts Black. The present cultivar is much more vigorous and resilient when compared with Platts Black, and remains a healthier plaint in more adverse conditions than Platts Black.

### SUMMARY OF THE INVENTION

Plants of the cultivar ‘FIT01’ have not been observed under all possible environmental and cultural conditions. The phenotype of the new cultivar may vary somewhat with variations in environment such as temperature, light intensity, length of day, irrigation amount and frequency, and/or fertilizer rate without, however, any variance in genotype.

The following traits have been repeatedly observed and are determined to be among the unique characteristics of ‘FIT01’. These characteristics distinguish ‘FIT01’ as a new and distinct cultivar from its closest comparator Phormium cookianum Platts Black:
1. Leaves are deep blackish purple all year; and
2. The foliage is more glossy and wider than Platts Black; and
3. The growth habit is taller than Platts Black and the leaf is wider.

### BRIEF DESCRIPTION OF THE DRAWINGS

The invention is depicted in the accompanying colored photographs in which:

FIG. 1 shows a potted plant illustrating the characteristic foliage.

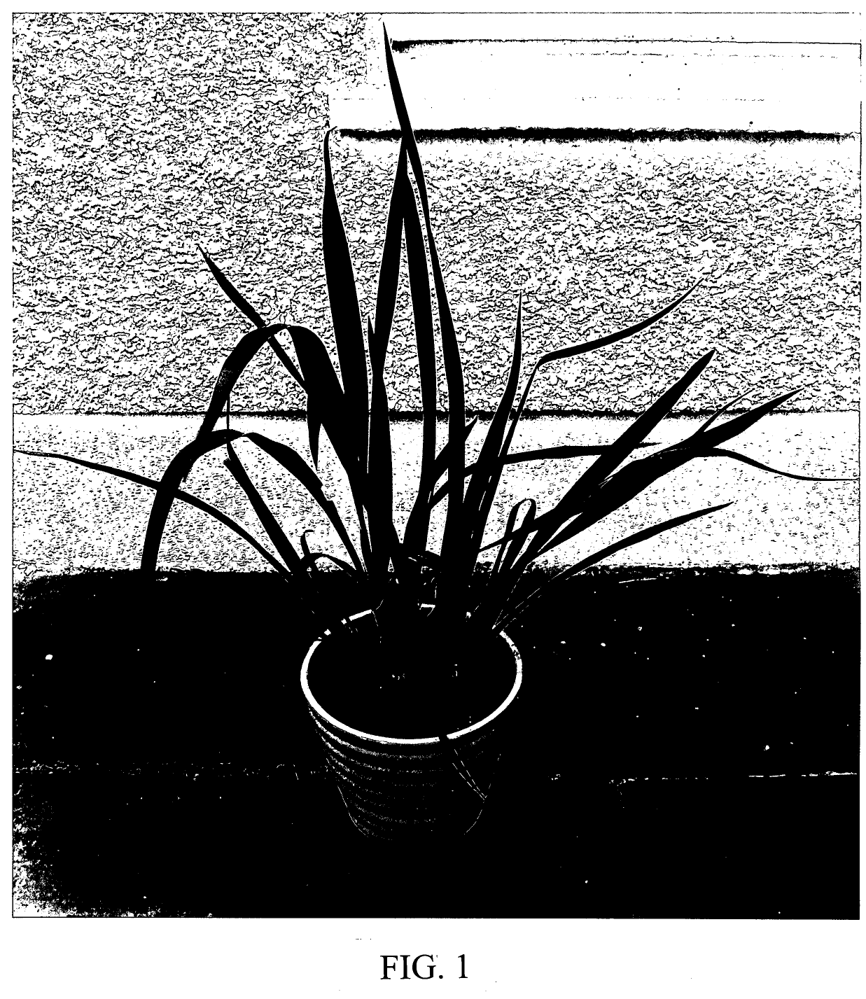

The photograph depicts the overall appearance of the new Phormium, showing the colors as true as it is reasonably possible to obtain in colored reproductions of this type. Colors in the photograph may differ slightly from the color values cited in the Detailed Botanical Description which more accurately describe the actual colors of the new Phormium.

### DETAILED BOTANICAL DESCRIPTION OF THE NEW PLANT

The following detailed description sets forth characteristics of the new cultivar. The data which defines each characteristic was collected from reproductions of the plant grown in a plastic greenhouse and outdoors.

In the following table of plant characteristics, color references are made to The Royal Horticultural Society of London Colour Chart, 1995 Edition.

### THE PLANT

Classification:
   Botanical.—Phormium cookianum.
   Varietal name.—‘FIT01’.
   Parentage.—Tissue Culture Mutation of Phormium cookianum Platts Black.
      Asexual propagation.—tissue culture mutation.
Form:
   Plant growth type.—Upright with drooping tips.
   Mean plant height.—1.5 M.
   Mean plant width.—1.5 M.
Leaves:
   Expanded leaf color upper.—Deep purple (R.H.S.) Leaf Shape — Wide lanceolate strap-like leaves Tip Shape — Acute Base Shape — Acuminate Margin — Entire.
   Leaf texture (upper).—Shiny, waxy surface.
   Venation pattern.—Parallel.
   Flower.—Flower development has not been observed on plants of the new Phormium.
   Lasting quality.—The Phormium is outstanding in wind and sun, as the foliage remains shiny and vibrant throughout the year. Providing the plant is not exposed to temperatures below −7° C., the plant will remain stable and colorful all year round.
   Disease resistance.—No known diseases have affected this plant to date.

### GENERAL CHARACTERISTICS

‘FIT01’ is primarily distinguished from other cultivars of Phormium genus by its deep blackish purple leaves having a glossy shine. ‘FIT01’ is an upright forming plant with a wide and tall growth habit.

What is claimed is:

1. A new and distinct Phormium cookianum plant named ‘FIT01’ substantially as illustrated and described.
